(19) United States
(12) Patent Application Publication
(10) Pub. No.: US 2021/0007886 A1
(43) Pub. Date: Jan. 14, 2021

(54) Facial Cold Compress Assembly

(71) Applicant: Helen Franks-Wilson, Grifton, NC (US)

(72) Inventor: Helen Franks-Wilson, Grifton, NC (US)

(21) Appl. No.: 16/506,529

(22) Filed: Jul. 9, 2019

(51) Int. Cl. A61F 7/02; A61F 5/08

(52) U.S. Cl. CPC A61F 7/0241; A61F 2007/0006; A61F 5/08

(57) ABSTRACT

A facial cold compress assembly for reducing facial swelling includes a strip that has a pair of pouches each being integrated into the strip. The strip is wearable on a user's face having the strip extending across the user's nose and beneath each of the user's eyes. In this way the strip is aligned with the user's nasal pathways. The strip is comprised of a thermally conductive material such that the strip is in thermal communication with the user's face when the strip is worn. An adhesive layer is bonded to the strip to retain the strip on the user's face. A pair of thermal packs is each removably positionable into a respective one of the pouches. Each of the thermal packs is positionable in a refrigerator for chilling and subsequently reducing swelling on the user's face and reduce congestion in the user's nasal pathways when the strip is worn.

### CROSS-REFERENCE TO RELATED APPLICATIONS

### Statement Regarding Federally Sponsored Research or Development

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to cold compress device and more particularly pertains to a new cold compress device for reducing facial swelling.

### (2) Description of Related Art Including Information Disclosed Under 37 CFR 1.97 and 1.98

The prior art relates to cold compress devices.

### BRIEF SUMMARY OF THE INVENTION

An embodiment of the disclosure meets the needs presented above by generally comprising a strip that has a pair of pouches each being integrated into the strip. The strip is wearable on a user's face having the strip extending across the user's nose and beneath each of the user's eyes. In this way the strip is aligned with the user's nasal pathways. The strip is comprised of a thermally conductive material such that the strip is in thermal communication with the user's face when the strip is worn. An adhesive layer is bonded to the strip to retain the strip on the user's face. A pair of thermal packs is each removably positionable into a respective one of the pouches. Each of the thermal packs is positionable in a refrigerator for chilling and subsequently reducing swelling on the user's face and reducing swelling congestion in the nasal pathways when the strip is worn.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top perspective view of a facial cold compress assembly according to an embodiment of the disclosure.

FIG. 2 is a bottom phantom view of an embodiment of the disclosure.

FIG. 3 is an exploded top view of an embodiment of the disclosure.

FIG. 4 is a cross sectional view taken along line 4-4 of FIG. 2 of an embodiment of the disclosure.

FIG. 5 is a perspective in-use view of an embodiment of the disclosure.

### DETAILED DESCRIPTION OF THE INVENTION

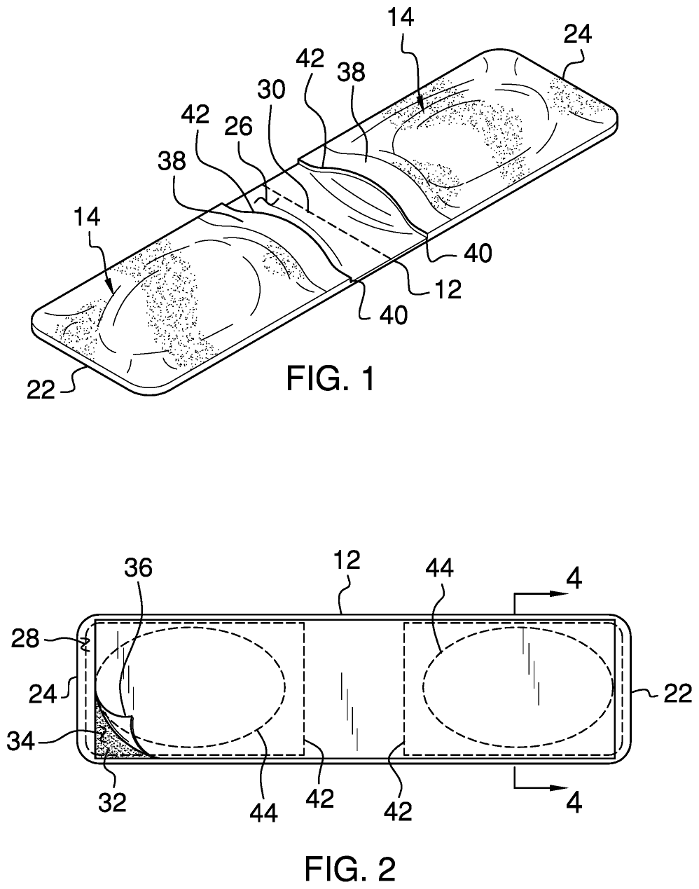

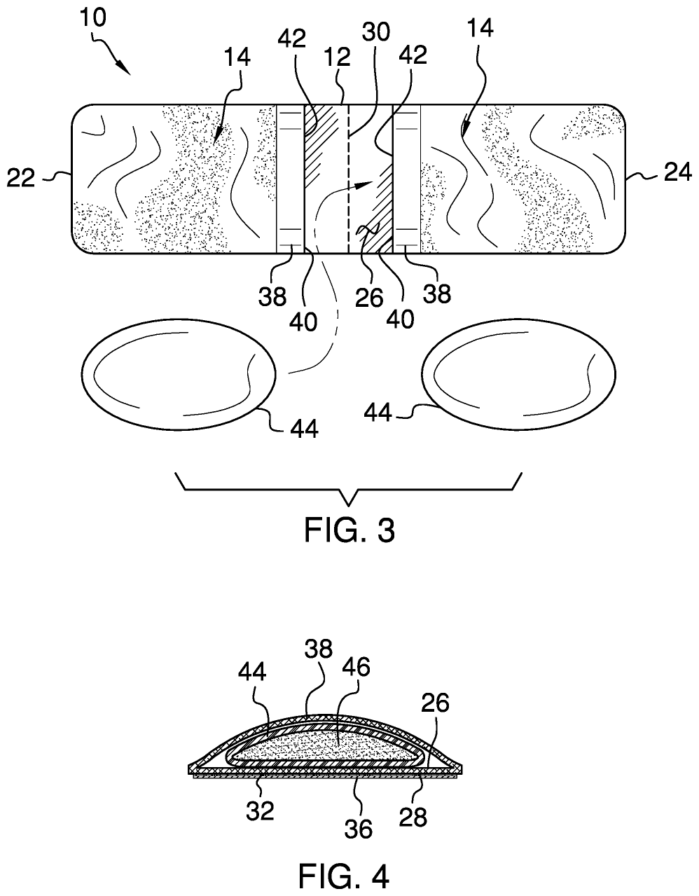

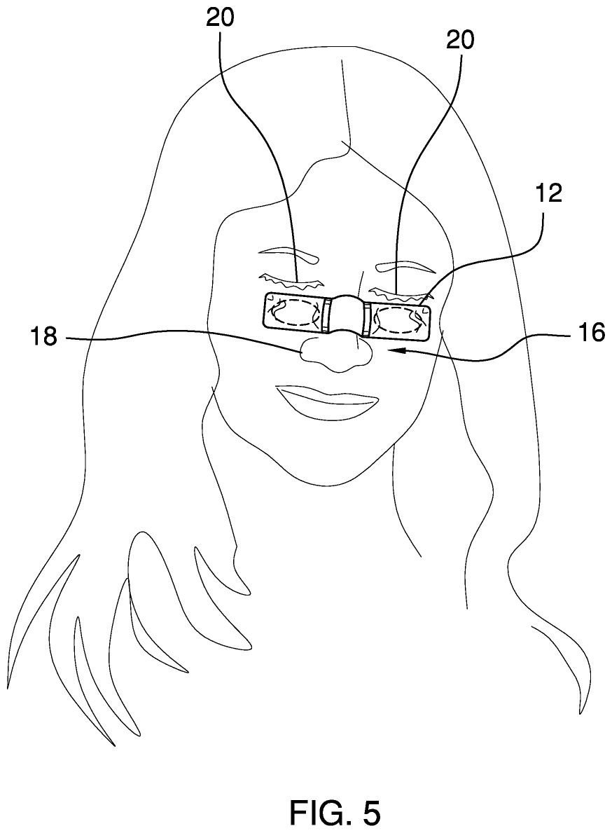

With reference now to the drawings, and in particular to FIGS. 1 through 5 thereof, a new cold compress device embodying the principles and concepts of an embodiment of the disclosure and generally designated by the reference numeral 10 will be described.

As best illustrated in FIGS. 1 through 5, the facial cold compress assembly 10 generally comprises a strip 12 that has a pair of pouches 14 each being integrated into the strip 12. The strip 12 is wearable on a user's face 16 having the strip 12 extending across the user's nose 18 and beneath each of the user's eyes 20. The strip 12 is comprised of a thermally conductive material such that the strip 12 is in thermal communication with the user's face 16 when the strip 12 is worn. The strip 12 has a first end 22, a second end 24, a top surface 26 and a bottom surface 28, and the strip 12 is elongated between the first 22 and second 24 ends. The strip 12 may have a length ranging between approximately 4.0 inches and 6.0 inches. Additionally, a plurality of perforations 30 may extend through the top 26 and bottom 28 surfaces of the strip 12 for dividing the strip 12 in half.

An adhesive layer 32 is bonded to the strip 12 and the adhesive layer 32 engages the user's face 16 when the strip 12 is worn. In this way the adhesive layer 32 retains the strip 12 on the user's face 16. The adhesive layer 32 covers the bottom surface 28 of the strip 12. The adhesive layer 32 has an exposed surface 34 with respect to the bottom surface 28 and the exposed surface 34 engages the user's face 16. The adhesive layer 32 may comprise a dermal adhesive or other type of adhesive that is safe for human skin. A protective sheet 36, such as laminated paper or the like, is removably positioned over the adhesive layer 32 for protecting the adhesive layer 32. The protective sheet 36 covers the exposed surface 34 of the adhesive layer 32.

A pair of covers 38 is each attached to the strip 12 for defining each of the pouches 14. The covers 38 are spaced apart from each other such that each of the covers 38 is positioned beneath a respective one of the user's eyes 20 when the strip 12 is worn. Each of the covers 38 has a perimeter edge 40 and the perimeter edge 40 of each of the covers 38 is coupled to the top surface 26 of the strip 12. The perimeter edge 40 of each of the covers 38 has a first side 42 and the first side 42 of the perimeter edge 40 of each of the covers 38 is open into an interior of a respective pouch 14. Each of the covers 38 extends inwardly from a respective first 22 and second 24 end of the strip 12 having the first side 42 associated with each of the covers 38 being spaced apart from each other.

A pair of thermal packs 44 is each removably positionable into a respective one of the pouches 14. Each of the thermal packs 44 is positionable in a refrigerator to chill the thermal packs 44. Each of the thermal packs 44 is in thermal communication with the strip 12 when the thermal packs 44 are positioned in the respective pouch 14. In this way each of the thermal packs 44 can reduce swelling on the user's face 16 and reduce congestion in the user's nasal pathways when the strip 12 is worn. Each of the thermal packs 44 is filled with a gelatinous thermal fluid 46 to enhance comfort for the user. Additionally, each of the thermal packs 44 is insertable through the first side 42 of the perimeter edge 40 of the respective cover 38.

In use, each of the thermal packs 44 is positioned in a refrigerator or the like to chill the thermal packs 44. Each of the thermal packs 44 is inserted into a respective one of the pouches 14 when the thermal packs 44 reach the desired temperature. The protective sheet 36 is removed from the adhesive layer 32 and the strip 12 is positioned to extend across the user's nose 18 and beneath each of the user's eyes 20. In this way each of the thermal packs 44 is positioned beneath each of the user eyes 20 for reducing swelling and puffiness beneath each of the user's eyes 20. Additionally, the thermal packs 44 reduce congestion and swelling in the user's nasal pathways. Each of the thermal packs 44 can be warmed prior to being inserted into the respective pouch 14 for applying heat therapy the user's face 16.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A facial cold compress assembly being configured to be worn beneath a user's eyes for treating swollen facial features around the eyes, said assembly comprising:
a strip having a pair of pouches each being integrated into said strip, said strip being wearable on a user's face wherein said strip is configured to extend across the user's nose and beneath each of the user's eyes having said strip being aligned with the user's nasal pathways, said strip being comprised of a thermally conductive material wherein said strip is configured to be in thermal communication with the user's face when said strip is worn;
an adhesive layer being bonded to said strip, said adhesive layer engaging the user's face when said strip is worn wherein said adhesive layer is configured to retain the strip on the user's face;
a pair of covers, each of said covers being attached to said strip for defining each of said pouches, said covers being spaced apart from each other wherein each of said covers is configured to be positioned beneath a respective one of the user's eyes when said strip is worn; and
a pair of thermal packs, each of said thermal packs being removably positionable into a respective one of said pouches, each of said thermal packs being positionable in a refrigerator wherein each of said thermal packs is configured to be chilled, each of said thermal packs being in thermal communication with said strip when said thermal packs are positioned in said respective pouch wherein each of said thermal packs is configured to reduce swelling on the user's face and reduce congestion in the user's nasal pathways when said strip is worn.

2. The assembly according to claim 1, wherein:
said strip has a first end, a second end, a top surface and a bottom surface, said strip being elongated between said first and second ends;
said adhesive layer covers said bottom surface of said strip, said adhesive layer having an exposed surface with respect to said bottom surface wherein said exposed surface is configured to engage the user's face; and
said assembly includes a protective sheet being removably positioned over said adhesive layer for protecting said adhesive layer, said protective sheet covering said exposed surface of said adhesive layer.

3. The assembly according to claim 2, wherein each of said covers has a perimeter edge, said perimeter edge of each of said covers being coupled to said top surface of said strip, said perimeter edge of each of said covers having a first side, said first side of said perimeter edge of each of said covers being open into an interior of a respective pouch, each of said covers extending inwardly from a respective first and second end of said strip having said first side associated with each of said covers being spaced apart from each other.

4. The assembly according to claim 2, wherein each of said thermal packs is filled with a gelatinous thermal fluid wherein each of said thermal packs is configured to enhance comfort for the user, each of said thermal packs being insertable through a first side of a perimeter edge of a respective cover.

5. A facial cold compress assembly being configured to be worn beneath a user's eyes for treating swollen facial features around the eyes, said assembly comprising:
a strip having a pair of pouches each being integrated into said strip, said strip being wearable on a user's face wherein said strip is configured to extend across the user's nose and beneath each of the user's eyes having said strip being aligned with the user's nasal pathways, said strip being comprised of a thermally conductive material wherein said strip is configured to be in thermal communication with the user's face when said strip is worn, said strip having a first end, a second end, a top surface and a bottom surface, said strip being elongated between said first and second ends;
an adhesive layer being bonded to said strip, said adhesive layer engaging the user's face when said strip is worn wherein said adhesive layer is configured to retain the strip on the user's face, said adhesive layer covering said bottom surface of said strip, said adhesive layer having an exposed surface with respect to said bottom surface wherein said exposed surface is configured to engage the user's face;
a protective sheet being removably positioned over said adhesive layer for protecting said adhesive layer, said protective sheet covering said exposed surface of said adhesive layer;
a pair of covers, each of said covers being attached to said strip for defining each of said pouches, said covers being spaced apart from each other wherein each of said covers is configured to be positioned beneath a respective one of the user's eyes when said strip is worn, each of said covers having a perimeter edge, said perimeter edge of each of said covers being coupled to said top surface of said strip, said perimeter edge of each of said covers having a first side, said first side of said perimeter edge of each of said covers being open into an interior of a respective pouch, each of said covers extending inwardly from a respective first and second end of said strip having said first side associated with each of said covers being spaced apart from each other; and
a pair of thermal packs, each of said thermal packs being removably positionable into a respective one of said pouches, each of said thermal packs being positionable in a refrigerator wherein each of said thermal packs is configured to be chilled, each of said thermal packs being in thermal communication with said strip when said thermal packs are positioned in said respective pouch wherein each of said thermal packs is configured to reduce swelling on the user's face and reduce congestion the user's nasal pathways when said strip is worn, each of said thermal packs being filled with a gelatinous thermal fluid wherein each of said thermal packs is configured to enhance comfort for the user, each of said thermal packs being insertable through said first side of said perimeter edge of said respective cover.
